(19) United States
(12) Patent Application Publication
(10) Pub. No.: US 2014/0303681 A1
(43) Pub. Date: Oct. 9, 2014

(54) TREATMENT OF SEXUAL DYSFUNCTION

(71) Applicants: Karim Khan, Atlanta, GA (US); Susheel Gundewar, Colleyville, TX (US)

(72) Inventors: Karim Khan, Atlanta, GA (US); Susheel Gundewar, Colleyville, TX (US)

(21) Appl. No.: 14/201,662

(22) Filed: Mar. 7, 2014

Related U.S. Application Data
(60) Provisional application No. 61/780,478, filed on Mar. 13, 2013.

(51) Int. Cl. A61N 1/36

(52) U.S. Cl. CPC A61N 1/36107
USPC 607/39; 607/116

(57) ABSTRACT

Systems for treating premature ejaculation include an electrical pulse generator and electrical leads in communication with sympathetic nerves serving ejaculatory structures. The system inhibits sympathetic nerve input to the ejaculatory structure with electrical impulses from the electrical pulse generator. The duration of inhibition may be predetermined or selectable. The system may be operated with user-actuated controls, a sensor and a timer, or combinations of these elements.

### BACKGROUND

The present disclosure relates to a surgically implantable biomedical device which operates as an ejaculation delaying system. This disclosure specifically relates to utilizing this device to affect the autonomic nerve function and supply to one or more human sex organs such as the epididymis, vas deferens, or the ejaculatory duct. These structures are included in the human ejaculatory system. The device prevents and/or delays the sympathetic nervous system stimulation to these organs which results in premature ejaculation (PE). In people experiencing PE and/or erectile dysfunction (ED), the device will cause a delay in ejaculation of semen and thereby delay orgasm for a specific amount of time allowing the person to complete his sexual experience. This therapeutic action of the device will offer a permanent treatment and cure for PE and prevention of concomitant ED in humans.

Premature ejaculation is defined by the International Society of Sexual Medicine as a male sexual dysfunction characterized by ejaculation which always or nearly always occurs prior to or within about one minute of vaginal penetration; and, inability to delay ejaculation on all or nearly all vaginal penetrations; and, negative personal consequences, such as distress, bother, frustration, and/or the avoidance of sexual intimacy. The median ejaculation latency time in patients with PE is about 6 minutes and causes unsatisfactory sexual experience for the patient and his partner.

PE is the most common form of sexual dysfunction in men. It has prevalence rates ranging from 20% to 30% among men aged 18-70 and the prevalence is similar across countries and age groups. It is also the most frequently underdiagnosed sexual condition because many physicians do not inquire about, nor patients mention, this form of sexual dysfunction. This may be due to factors such as the lack of effective therapies or patients' reluctance to present PE as a medical complaint since it is perceived as sign of loss of virility. It is estimated that only 9% of the patients suffering from this condition seek medical help.

It appears that PE has no single etiology although genetic, environmental, endocrine and psychological factors have all been implicated. Treatments have been based on both its neurophysiologic modulation and behavioral modifications. However, there are currently no therapies for the treatment of PE which are approved by the U.S. Food and Drug Administration. Certain medications that have been used to treat this condition include off-label use of selective serotonin reuptake inhibitors, tricyclic antidepressants, phosphodiesterase type 5 inhibitors, and topical anesthetics. These drugs are often associated with severe adverse effects and often lack efficacy. No evidence suggests that the use of these medications would lead to a cure of PE. Behavioral techniques have been the mainstay of PE treatment, and include techniques to decrease sensory input and exercises to strengthen pelvic floor musculature; however these methods have been largely shown to be ineffective at curing this condition.

PE can have a significant impact on the quality of life of the patient and his sexual partner, and may lead to psychological distress and loss of self-esteem. It has devastating effects on sexual relationships and often leads to complete lack of sexual intimacy. The inability of a male to satisfy his sexual partner during intercourse leads to disappointment and unfulfilled desires of intimacy. Single men with PE tend to avoid sexual intercourse and thus this condition serves as a barrier to developing new relationships. Men with PE are anxious and tend not to participate in foreplay which is viewed by their partner as a sign of rejection or lack of interest. This often leads to anger, strain and frustration in their relationship. This can further exacerbate the condition and lead to a vicious cycle that perpetuates the problem of PE.

Importantly, several studies of the epidemiologic society have shown a strong correlation between PE and ED. A survey conducted by the Global Study of Sexual Attitudes and Behaviors (GSSAB) suggested that 41% of men who reported ED also complained of PE, and 30% of men who reported PE also experienced ED. This suggests that both ED and PE may share similar pathophysiology. In contrast to ED which begins in a later stage of life, PE is often seen in young adults and begins at an early age. Experiencing PE during early adulthood can eventually lead to ED between the third and fourth decade of life. ED in these patients is caused as a consequence of PE and may be related to the same psychosocial and behavioral factors associated with PE.

There are several structures that constitute the ejaculatory system in men.

Seminiferous tubules. The production of sperm occurs in the testis following which the spermatozoa are transported out of the testis and into the epididymis through a series of efferent ductules also called the seminiferous tubules.

Epididymis. The seminiferous tubules join together to form the epididymis which is a tubular structure that is about 5 cm in length and coiled on the posterior surface of each testis. The epididymis functions as the site where maturation of sperm takes place. This is also a site to store sperm until the next ejaculation. The epididymis is made of the head which is the proximal part of the epididymis and receives sperm from the testis, the body, which is the highly convoluted middle part of the epididymis where maturation of sperm takes place, and the tail, which is responsible for carrying the sperm to the ductus deferens. The contraction of smooth muscle in the wall of the epididymis propels the sperm into the ductus deferens.

Ductus Deferens. The ductus deferens or the sperm duct arises from the epididymis in the scrotum and runs alongside the testicular blood vessels and nerves in the spermatic cord into the abdominal cavity through the inguinal canal. The inguinal canal is an opening in the abdominal wall for the spermatic cord. The smooth muscle layer of the sperm duct contracts in waves of peristalsis during ejaculation.

Seminal Vesicles. These are tubular glands that are located within the pelvis posterior to the urinary bladder. The two seminal vesicles secrete alkaline fluid that provides an energy source for sperm and enhances sperm mobility. The duct of each seminal vesicle joins the ductus deferens on that side to form the ejaculatory duct.

Ejaculatory Ducts. There are two ejaculatory ducts. Each receives sperm from the ductus deferens and the secretions of the seminal vesicle on its own side. Both ejaculatory ducts empty into the single urethra.

Prostate Gland. The prostate gland is a muscular gland that surrounds the urethra as it emerges from the bladder. The smooth muscle of the prostate gland contracts during ejaculation to contribute to the expulsion of semen from the urethra.

Bulbourethral Glands. The bulbourethral glands, also called Cowper's glands, are located below the prostate gland and empty into the urethra. They secrete a clear viscous fluid that helps to lubricate the urethra prior to ejaculation.

Penis and Urethra. The penis is an external genital organ which consists of a layer of smooth muscle and connective tissue. The urethra is the last part of the urinary tract and traverses through the spongy layer of the penis for the passage of urine and for the ejaculation of semen.

Spermatogenesis takes place inside a male's testes, specifically in the walls of the seminiferous tubules. The epididymis receives immature sperm from the testis and stores it for several days where they mature. When ejaculation occurs, sperm is forcefully expelled from the tail of the epididymis into the ductus deferens. Sperm travels through the ductus deferens and up the spermatic cord into the pelvic cavity, over the ureter to the prostate behind the bladder. Here, the vas deferens joins with the seminal vesicle to form the ejaculatory duct, which passes through the prostate and empties into the urethra. Upon the sperm's exit from the testes, into the vas deferens, muscular movements take over. When ejaculation occurs, rhythmic muscle movements of peristalsis propel the sperm forward.

The reproductive system in humans is supplied by somatic and autonomic nerves.

Somatic nerves are myelinated, larger in diameter, and rapidly conduct neural impulses. The somatic sensory nerves mediate information from skin, skeletal muscles, and joints, while motor nerves mediate impulses to skeletal muscles. The somatic sensory and motor innervation of the male genitalia is carried by the branches of the pudendal nerve and its motor functions are under voluntary control.

Autonomic fibers or nerves are small diameter and non-myelinated fibers that conduct neural impulses relatively slowly. Their motor impulses mediate involuntary responses such as sweat gland release, blood flow, and gut peristalsis. Autonomic sensory fibers are known as visceral afferents, and they conduct sensory information from the viscera and blood vessels to the central nervous system (CNS). The pelvic plexus is the main crossroad for genital autonomic nerves, consisting of parasympathetic fibers from the sacral nerve roots (S2-S4) and sympathetic nerve fibers from the thoracolumbar sympathetic nerve roots (T10-L2). The pelvic plexus lies on either side of the bladder and rectum, and supplies all the pelvic viscera, including the bladder, urethral sphincter, and the male genital tissue.

The male sexual response, and more specifically the ejaculatory response, is considered a reflex event in which there is an involuntary response to sexual stimulus via the reflex arc. Several excitatory signals (afferent arms) conduct sensory stimuli to the central nervous system from the special senses (eyes, ears), non-genital erogenous areas (nipples, neck), and from the genitals. The resultant responses (efferent arms) can be emotional and psychological changes (pleasure), systemic non-genital responses (increase in heart rate and respiration) and genital changes (erection, ejaculation).

The male sexual response can be considered as three events: erection, ejaculation, and orgasm. Each of these can be considered as part of a reflex. The autonomic nerves supply both sympathetic and parasympathetic nerves to the various organs of the reproductive system that work in unison for the male sexual response. The parasympathetic nerves arising from S2-S4 sacral nerve roots primarily mediate the erection phase and the sympathetic nerves arising from the T10-L210 nerve roots mediate the ejaculation phase of the sexual response.

The ejaculation phase consists of both emission and expulsion of semen and is mediated via alpha adrenergic receptors. Emission is the term used when sperm moves into the urethra. Ejaculation is the term used when sperm is forced out of the urethra and the penis. These are both stimulated by sympathetic nerves. Indeed drugs such as tamsulosin, which is an alpha 1 adrenergic receptor blocker used in the treatment of hypertension and benign prostate hypertrophy, cause smooth muscle cell relaxation of the vas deferens, seminal vesicles and prostate and thus cause delayed ejaculation. However, due to the systemic effects of these medications which include hypotension etc., these drugs have not been widely used for the treatment of PE. However, non-drug therapies that aim to prevent or delay the sympathetic conduction to the organs involved in ejaculation may provide an effective treatment for PE.

### BRIEF DESCRIPTION OF THE DRAWINGS

While exemplary embodiments of the present technology have been shown and described in detail below, it will be clear to the person skilled in the art that changes and modifications may be made without departing from its scope. As such, that which is set forth in the following description and accompanying drawings is offered by way of illustration only and not as a limitation. The actual scope of the invention is intended to be defined by the following claims, along with the full range of equivalents to which such claims are entitled.

In addition, one of ordinary skill in the art will appreciate upon reading and understanding this disclosure that other variations for the technology described herein can be included within the scope of the present technology.

In the following Detailed Description, various features are grouped together in several embodiments for the purpose of streamlining the disclosure. This method of disclosure is not to be interpreted as reflecting an intention that exemplary embodiments of the technology require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

Identical reference numerals do not necessarily indicate an identical structure. Rather, the same reference numeral may be used to indicate a similar feature or a feature with similar functionality Not every feature of each embodiment is labeled in every figure in which that embodiment appears, in order to keep the figures clear. Similar reference numbers (e.g., those that are identical except for the first numeral) are used to indicate similar features in different embodiments.

FIG. 1 is a diagram of the sympathetic and parasympathetic nervous systems, the sympathetic nerves shown in dashed lines, the parasympathetic nerves shown in solid lines;

FIG. 2 is a diagram of pelvic nerves superimposed on an anatomical detail view of male pelvic organs;

FIG. 3 is a block diagram of a nerve inhibition device;

FIG. 4 is a diagram of pelvic nerves and a nerve inhibition device superimposed on an anatomical detail view of male pelvic organs, the nerve inhibition device connected to a nerve at a first inhibition site;

FIG. 5 is a diagram of pelvic nerves and a nerve inhibition device superimposed on an anatomical detail view of male pelvic organs, the nerve inhibition device connected to a nerve at a first inhibition site;

FIG. 6 is a diagram of pelvic nerves and a nerve inhibition device superimposed on an anatomical detail view of male pelvic organs, the nerve inhibition device connected to a nerve at a first inhibition site;

FIG. 7 is a diagram of pelvic nerves and a nerve inhibition device superimposed on an anatomical detail view of male pelvic organs, the nerve inhibition device connected to a nerve at a first inhibition site;

FIG. 8 is a diagram of pelvic nerves and a nerve inhibition device superimposed on an anatomical detail view of male pelvic organs, the nerve inhibition device connected to a nerve at a first inhibition site;

FIG. 9 is a block diagram of another nerve inhibition device; and

FIG. 10 is a block diagram of a method of use of a nerve inhibition device

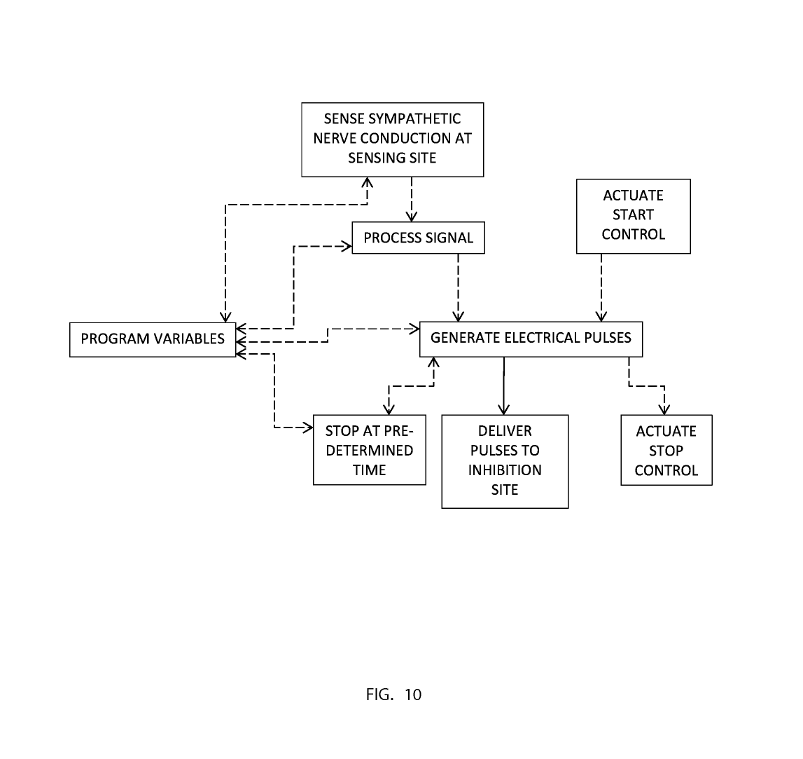

### DETAILED DESCRIPTION

Standard medical planes of reference and descriptive terminology are employed in this specification. A sagittal plane divides a body into right and left portions. A mid-sagittal plane divides the body into equal right and left halves. A coronal plane divides a body into anterior and posterior portions. A transverse plane divides a body into superior and inferior portions. Anterior means toward the front of the body. Posterior means toward the back of the body. Superior means toward the head. Inferior means toward the feet. Medial means toward the midline of the body. Lateral means away from the midline of the body. Axial means toward a central axis of the body. Abaxial means away from a central axis of the body.

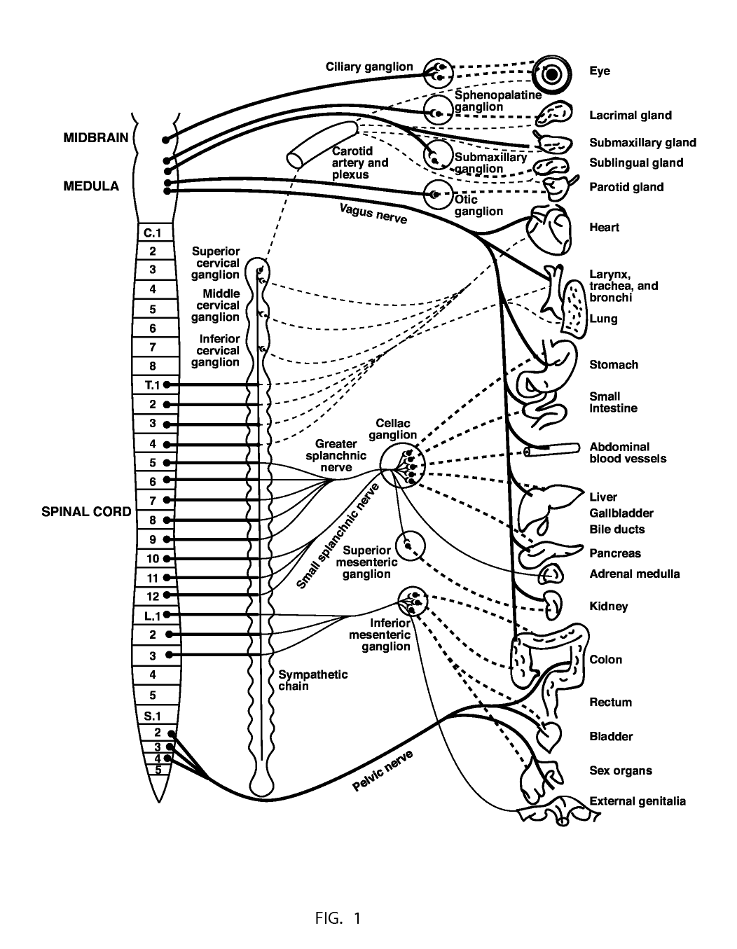

Referring to FIG. 1, the autonomic nervous system is shown diagrammatically.

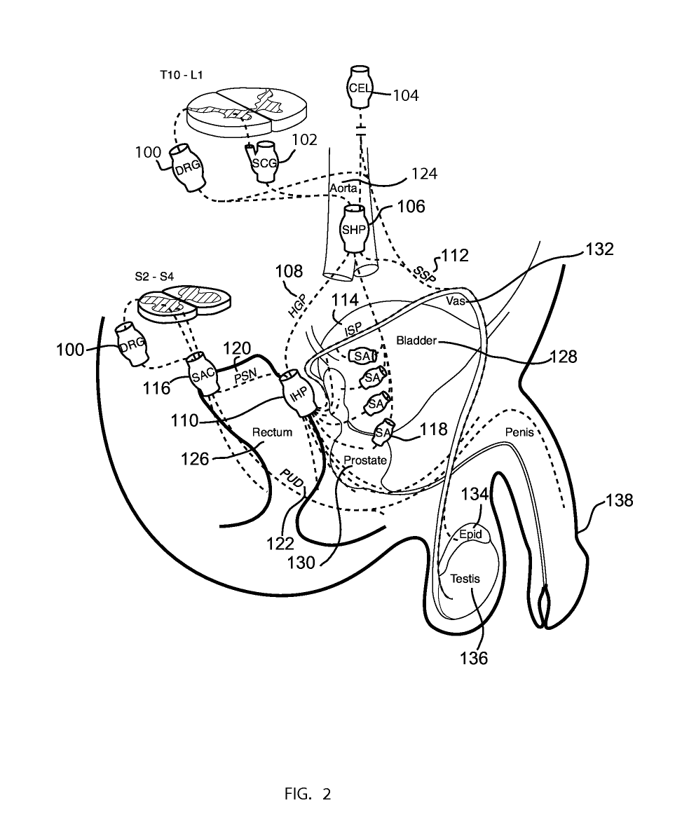

Referring to FIG. 2, T10-L1 refers to the portion of the spine which includes tenth through twelfth thoracic vertebrae and a first lumbar vertebra. The sympathetic nerves which primarily mediate the ejaculation phase arise from T10-L1 nerve roots. S2-S4 refers to another portion of the spine which includes second through fourth sacral vertebrae. The parasympathetic nerves which primarily mediate the erection phase arise from S2-S4 nerve roots.

Autonomic neural structures identified in FIG. 2 include the dorsal root ganglion (DRG) 100; sympathetic chain ganglion (SCG) 102; celiac plexus (CEL) 104; superior hypogastric plexus (SHP) 106; hypogastric plexus (HGP) 108; inferior hypogastric plexus (IHP) 110; superior spermatic plexus (SSP) 112; inferior spermatic plexus (ISP) 114; sacral plexus (SAC) 116; short adrenergic projections (SA) 118; pelvic splanchnic nerve (PSN) 120; and pudendal nerve (PUD) 122. Other neural structures are shown but not identified, although these structures may be identified readily with the help of an anatomical text.

Organs identified in FIG. 2 include the aorta 124, rectum 126, bladder 128, prostate 130, vas deferens 132, epididymis 134, testis 136, and penis 138. Other organs and tissue structures are shown but not identified, although these structures may be identified readily with the help of an anatomical text.

The technology disclosed herein provides comprehensive systems for inhibiting and/or delaying nerve conduction through the sympathetic neurons that arise from the thoracolumbar region (T10 to L2) spinal cord. Delaying or inhibiting the conduction of the sympathetic nervous system arising from T10 to L2 spinal cord level may be an effective treatment and cure for people with PE and may prevent concomitant ED.

Neuronal stimulation regulated by the central nervous system is conducted through the spinal centers and, when trigged, causes peripheral autonomic nerve stimulation via sympathetic neurons to promote ejaculation. The device may inhibit sympathetic nerve conduction from reaching to the targeted sex organs during sexual arousal or sexual intercourse. The device may also sense sympathetic nerve conduction to the ejaculatory structures, which may trigger activation of the device. On the other hand, the device may be activated by a user actuating a control located, for example, on a remote control device.

The treatment provided by the device may be carried out by blocking two or more sympathetic nerves arising from T10 to L2 spinal cord level, including the pelvic plexus, at the time of sexual excitation to inhibit the electrical impulses to the ejaculatory structures.

The device is a treatment apparatus that contains inhibition leads adapted for placement on sympathetic nerves which serve the ejaculatory structures. The device may also include sensing leads for similar placement. It may also contain a sensory unit that may activate an inhibiting signal generator for generating inhibitory impulses at signal frequencies necessary to electrically inhibit nerve impulses to those sites completely.

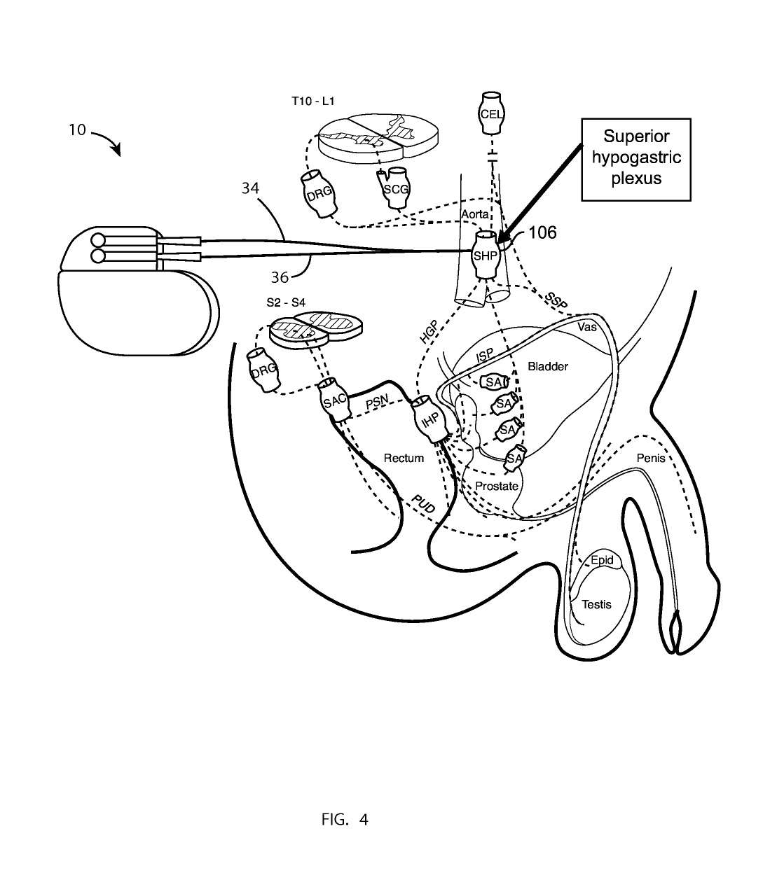

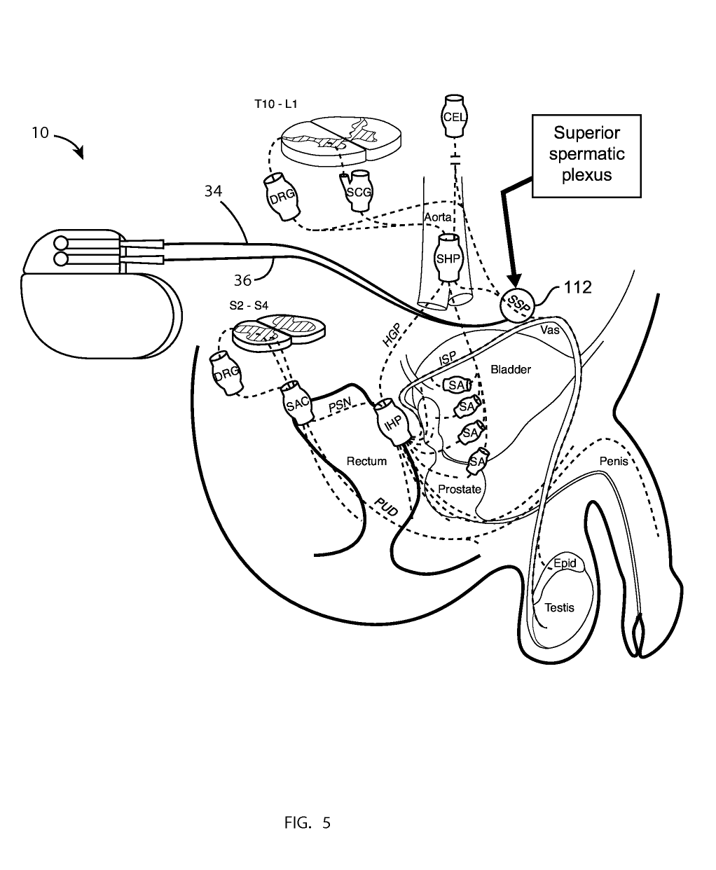

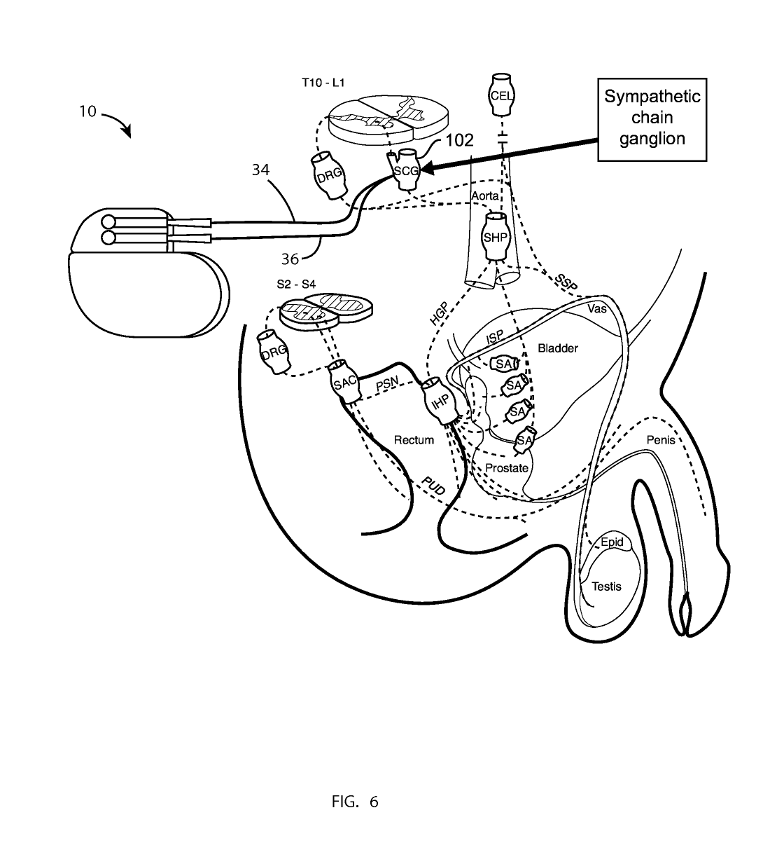

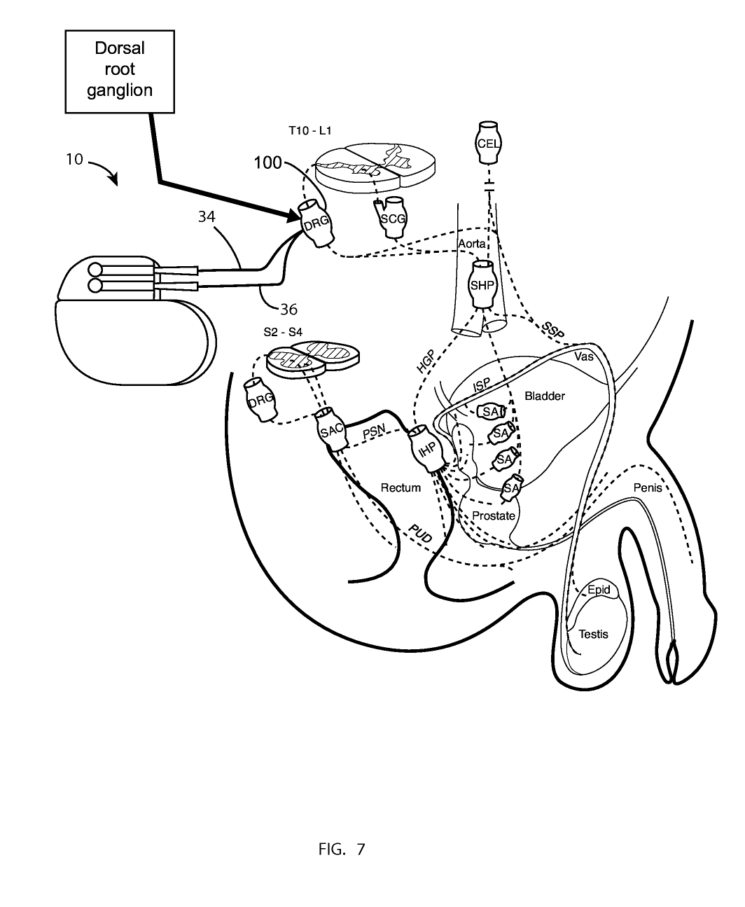

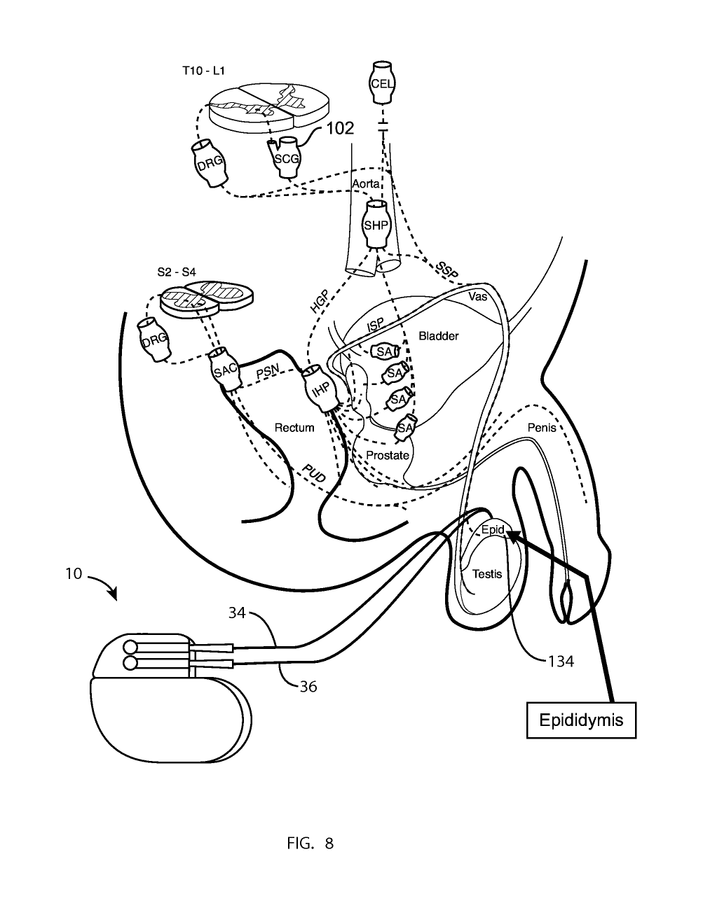

The leads from the device may be connected to one or more of the superior hypogastric plexus (SHP) (FIG. 4), superior spermatic plexus (SSP) (FIG. 5), sympathetic chain ganglion (SCG) (FIG. 6), dorsal root ganglion (DRG) (FIG. 7), and/or celiac plexus (CEL). Thoracic nerves ten, eleven and twelve (T10, T11, T12) and lumbar nerves one and two (L1, L2) of the sympathetic chain ganglion (SCG) may be connected to one or more leads from the device in addition to, or instead of, the preceding neural structures. Optionally, one or more leads from the device may be connected to a sex organ directly such as the epididymis (FIG. 8), vas deferens, and/or ejaculatory duct in addition to, or instead of any of the preceding neural structures. A direct connection may decrease and/or inhibit the normal reflexive neural stimulation of these target organs leading to a delay in ejaculation.

It will be appreciated that there are a variety of nerve conduction sensors and inhibitors that are commercially available for implantation and which include leads to connect directly to nerves.

The duration of inhibitory signals from the device may be programmable. For example, the device may be programmed via an external programming unit by a physician or other health care provider based on user preferences. A sustained electrical charge through the device at the onset of sexual activity will prevent PE for the programmed length of time. After the preset time duration, the device will automatically deactivate once the preset device activation time is achieved. After the device is deactivated, ejaculation is free to occur. The device will then remain off until it is reactivated by the sympathetic nerve activity prior to next sexual activity.

In another example, the duration for which the device is activated may be under the control of the user. This arrangement may enable the user to inhibit nerve conduction through the sympathetic nerves responsible for ejaculatory response.

The device and all of its components except for the remote control device may be surgically implanted under the skin of the beneficiary and may not be exposed outside of the beneficiary's body or skin. After the implantation of the device and its components, the skin may be fully sutured by the surgeon at the time of implantation.

The device may have a battery contained within its body. The fully charged battery may have a service life of several years in order to provide sufficient time to achieve the goals of treatment. For example, the battery may have a 5 year service life.

The implanted device may weight 20 gm. The body size may be around 15 square cm. The size and length of the leads of the device which connect the device to nerves and the target organs in the beneficiary's body may vary. Such a size and length of the leads may be determined and chosen by the surgeon at the time of implantation of the device.

In patients with premature ejaculation, the device may be implanted under the skin of the beneficiary for as long as necessary to permit the user to achieve a delay in ejaculation that consistently permits a sexually gratifying experience. The device may be explanted upon satisfactory conclusion of the treatment.

The present disclosure provides a treatment and cure for PE and prevention of ED. The implantation of the system will include one device and one or more leads under the skin of the beneficiary which will be done with a minimally invasive surgical manner The benefits of the device include cure and treatment of PE, preventing ED, physically and mentally satisfying sexual intercourse, and maintaining a healthy relationship as whole.

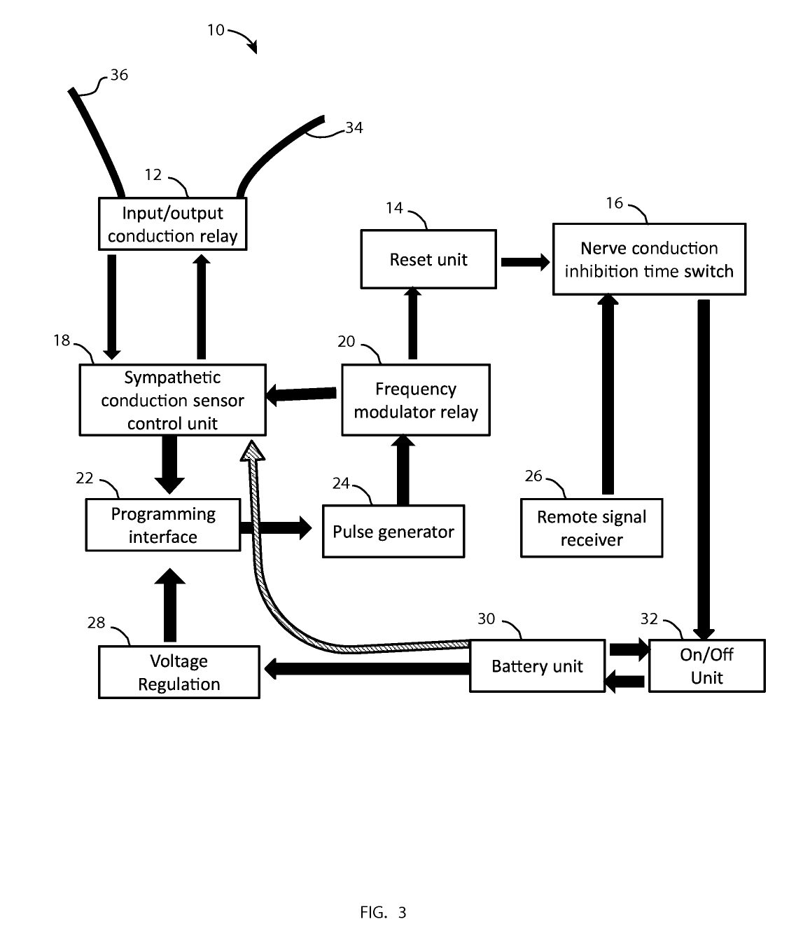

Referring to FIG. 3, a block diagram illustrates one possible arrangement of components in a treatment system 10. In this example, the system 10 includes an input/output conduction relay 12, a reset unit 14, a nerve conduction inhibition time switch 16, a sympathetic conduction sensor control unit 18, a frequency modulator relay 20, a programming interface 22, a pulse generator 24, a remote signal receiver 26, a voltage regulator 28, a battery unit 30, and an on/off unit 32. Systems that include fewer than these listed components are also contemplated.

A lead 34 is connected to the device via the input/output conduction relay 12. This example also shows a second lead 36 connected to the input/output conduction relay 12. The input/output conduction relay 12 is a two-way conduction communication unit. The input/output conduction relay 12 receives the sympathetic conduction signal from the body of the beneficiary through sensing lead 34 during sexual arousal and/or ejaculation and sends the electrical pulses generated by the device exit back to the target inhibition area through inhibition lead 36 for inhibiting the sympathetic nerve conduction. The input/output conduction relay 12 also sends the sympathetic conduction signal to the sympathetic conduction sensor control unit 18.

The sympathetic conduction sensor control unit 18 senses the sympathetic conduction signal from the input/output conduction relay 12 and controls the corresponding outflow of electrical impulses. The sympathetic conduction sensor control unit 18 is also responsible for controlling and regulating the electrical impulses generated by the device for the inhibition of sympathetic nerve conduction during premature ejaculation.

Once the sympathetic conduction signal passes the conduction sensor control unit the signal then enters the programming interface 22. This unit then sends a signal to alert the pulse generator 24.

Once the pulse generator 24 receives the signal from the programming interface unit 22, it is ready to generate and send out the appropriate signals to the target inhibition area via the inhibition lead 36 connected to the device by input/output unit 12 for causing sympathetic nerve conduction inhibition.

The remote signal receiver 26 may be used to change the device on/off time remotely by the physician. The remote control unit may be located in the doctor's office for adjusting the time for the preference of the beneficiary. However, in other examples, the user may have a remote control unit at home with which to reprogram the on/off time.

The frequency modulator relay 20 keeps track of the signals out flow to the target inhibition area in the body so the device 10 will only deliver the right amplitude of electrical impulses.

The reset unit 14 communicates between the time switch 16 and programming interface 22 to keep track and allow the device to turn ON and OFF according the programmed timing.

The nerve conduction inhibition time switch 16 triggers by the programming interface 22 via the reset unit 14. This then allows the ON/OFF unit 32 to turn on or off the device without user input or actuation of a control.

Once the On/Off unit 32 gets the command from the programming interface 22 where the programming interface 22 activates by sympathetic nerve conduction generated the sympathetic nervous system inside the beneficiary's body, the On/Off unit 32 right away executes the command by turning on or off the device.

The voltage regulator 28 gives the device 10 the proper Direct Current (DC) and keeps the amplitude of the current the same throughout the running time of the device.

FIGS. 4-8 illustrate the treatment system 10 electrically coupled to various sensing and/or inhibition sites. The leads 34, 36 from the device 10 are shown connected to the superior hypogastric plexus (SHP) 106 (FIG. 4), superior spermatic plexus (SSP) 112 (FIG. 5), sympathetic chain ganglion (SCG) 102 (FIG. 6), dorsal root ganglion (DRG) 100 (FIG. 7), and epididymis 134 (FIG. 8). Any of the treatment systems disclosed herein may be substituted for system 10 in FIGS. 4-8.

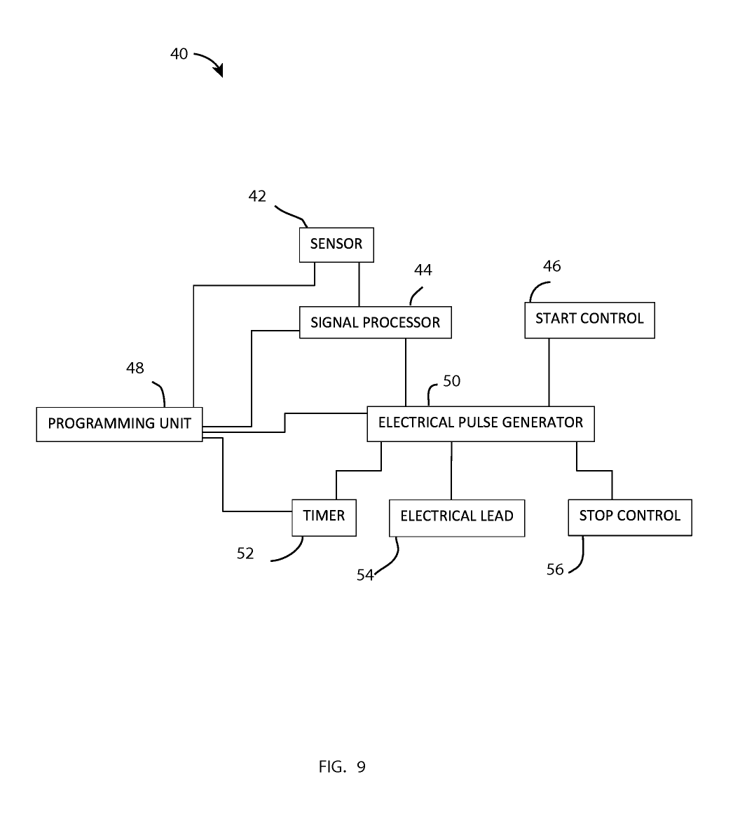

FIG. 9 illustrates another block diagram for a treatment system 40 which includes a sensor 42, a signal processor 44, a start control 46, a programming unit 48, an electrical pulse generator 50, a timer 52, an electrical lead 54, and a stop control 56. These components may be assembled in various configurations and/or operated according to different methods, each of which may be considered another example of a system according to the present disclosure.

A first example system may be for treating PE, and may include the electrical pulse generator 50 and the electrical lead 54. When the electrical lead 54 is in electrical communication with a sympathetic nerve serving an ejaculatory structure, this system may inhibit sympathetic nerve input to the ejaculatory structure with electrical impulses from the electrical pulse generator 50. Variations of this system may inhibit sympathetic nerve input for a selected amount of time. A selected amount of time may be provided in systems where a user manually actuates the stop control 56 to stop the inhibition Inhibition may be initiated automatically by the sensor 42 combined with the signal processor 44, or manually by the user actuating the start control 46. However, in all of these situations, the length of inhibition time is controlled by the user. Other variations of this system may inhibit sympathetic nerve input for a predetermined amount of time. A predetermined amount of time may be provided in systems where the timer 52 automatically shuts off the inhibition Inhibition may be initiated automatically by the sensor 42 combined with the signal processor 44, or manually by the user actuating the start control 46. However, in all of these situations, the length of inhibition time is controlled by the timer 52.

A second example system may be for treating PE, and may include the electrical pulse generator 50 and the electrical lead 54. When the electrical lead 54 is in electrical communication with a sympathetic nerve serving an ejaculatory structure, this system may inhibit sympathetic nerve input to the ejaculatory structure with electrical impulses from the electrical pulse generator 50 for a selected amount of time. As above, a selected amount of time may be provided when a user manually actuates the stop control 56 to stop the inhibition. Inhibition may be initiated automatically by the sensor 42 combined with the signal processor 44, or manually by the user actuating the start control 46.

A third example system may also be for treating PE, and may include the electrical pulse generator 50 and the electrical lead 54. When the electrical lead 54 is in electrical communication with a sympathetic nerve serving an ejaculatory structure, this system may inhibit sympathetic nerve input to the ejaculatory structure with electrical impulses from the electrical pulse generator 50 for a predetermined amount of time. As above, a predetermined amount of time may be provided in systems where the timer 52 automatically shuts off the inhibition. One variation of this system provides a closed loop feedback system, with the sensor 42 controlling initiation of inhibition, and the timer 52 controlling termination of inhibition.

In these example systems, a frequency, amplitude, wave form, or other characteristic of the electrical impulses may be programmable. The sensor 42 may have a programmable sensitivity. The timer may have a programmable duration. Programming may be done by a health care provider or by the user. The start control 46 and stop control 56 (if present) may be on a remote control or accessible through a computer application. Instead of separate controls, a single two-state start/stop control may be provided, like a toggle switch or slider.

The components disclosed herein may be made from metals, polymers, ceramics, glasses, composite materials, biological materials or tissues, insulators, conductors, semiconductors, or other biocompatible or non-biocompatible materials. Different materials may be used for individual components. Different materials may be combined in a single component.

It should be understood that the present system, kits, apparatuses, and methods are not intended to be limited to the particular forms disclosed. Rather, they are to cover all combinations, modifications, equivalents, and alternatives falling within the scope of the claims.

The claims are not to be interpreted as including means-plus- or step-plus-function limitations, unless such a limitation is explicitly recited in a given claim using the phrase(s) “means for” or “step for,” respectively.

The term “coupled” is defined as connected, although not necessarily directly, and not necessarily mechanically.

The use of the word “a” or “an” when used in conjunction with the term “comprising” in the claims and/or the specification may mean “one,” but it is also consistent with the meaning of “one or more” or “at least one.” The term “about” means, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed.

In the foregoing Detailed Description, various features are grouped together in several embodiments for the purpose of streamlining the disclosure. This method of disclosure is not to be interpreted as reflecting an intention that the embodiments of the invention require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

1. A system for treating premature ejaculation, comprising:
an electrical pulse generator; and
an electrical lead;
wherein, when the electrical lead is in electrical communication with a sympathetic nerve serving an ejaculatory structure, the system inhibits sympathetic nerve input to the ejaculatory structure with electrical impulses from the electrical pulse generator.

2. The system of claim 1, wherein a frequency of the electrical impulses is programmable.

3. The system of claim 1, wherein the system inhibits sympathetic nerve input for a selected amount of time.

4. The system of claim 3, comprising:
a start control; and
a stop control;
wherein actuation of the start control initiates the electrical impulses, wherein actuation of the stop control terminates the electrical impulses.

5. The system of claim 4, wherein the start and stop controls are on a remote control unit.

6. The system of claim 4, wherein the start and stop controls are combined in a single stop/start control.

7. The system of claim 3, comprising:
a sensor;
a processor; and
a stop control;
wherein, when the sensor is in electrical communication with a sympathetic nerve serving an ejaculatory structure and a threshold conduction level of the sympathetic nerve occurs, the sensor sends a sensor signal to the processor upon sensing the threshold conduction of the nerve;
wherein the processor emits a processor signal to the electrical pulse generator upon receiving the sensor signal;
wherein the processor signal initiates the electrical impulses, wherein actuation of the stop control terminates the electrical impulses.

8. The system of claim 7, wherein a sensitivity of the sensor to the threshold conduction level of the sympathetic nerve is programmable.

9. The system of claim 7, wherein the stop control is on a remote control unit.

10. The system of claim 1, comprising:
a timer;
wherein the system inhibits sympathetic nerve input for a predetermined amount of time;
wherein the timer stops the electrical pulse generator when the predetermined amount of time has occurred.

11. The system of claim 10, wherein the predetermined amount of time is programmable.

12. The system of claim 10, comprising:
a start control;
wherein actuation of the start control initiates the electrical impulses.

13. The system of claim 12, wherein the start control is on a remote control unit.

14. The system of claim 10, comprising:
a sensor; and
a processor;
wherein, when the sensor is in electrical communication with a sympathetic nerve serving an ejaculatory structure and a threshold conduction level of the sympathetic nerve occurs, the sensor sends a sensor signal to the processor upon sensing the threshold conduction of the nerve;
wherein the processor emits a processor signal to the electrical pulse generator upon receiving the sensor signal;
wherein the processor signal initiates the electrical impulses.

15. The system of claim 14, wherein a sensitivity of the sensor to the sympathetic nerve is programmable.

16. A system for treating premature ejaculation, comprising:
a electrical pulse generator; and
an electrical lead;
wherein, when the electrical lead is in electrical communication with a sympathetic nerve serving an ejaculatory structure, the system inhibits sympathetic nerve input to the ejaculatory structure with electrical impulses from the electrical pulse generator for a selected amount of time.

17. The system of claim 16, wherein a frequency of the electrical impulses is programmable.

18. The system of claim 16, wherein actuation of a start control initiates the electrical impulses, wherein actuation of a stop control terminates the electrical impulses.

19. The system of claim 18, wherein the start and stop controls are on a remote control unit.

20. The system of claim 18, wherein the start and stop controls are combined in a single stop/start control.

21. The system of claim 16, wherein a sensor initiates the electrical impulses, wherein actuation of a stop control terminates the electrical impulses.

22. The system of claim 21, wherein a sensitivity of the sensor is programmable.

23. The system of claim 21, wherein the stop control is on a remote control unit.

24. A system for treating erectile dysfunction, comprising:
an electrical pulse generator; and
an electrical lead;
wherein, when the electrical lead is in electrical communication with a sympathetic nerve serving an ejaculatory structure, the system inhibits sympathetic nerve input to the ejaculatory structure with electrical impulses from the electrical pulse generator for a predetermined amount of time.

25. The system of claim 24, wherein a frequency of the electrical impulses is programmable.

26. The system of claim 24, wherein the predetermined amount of time is programmable.

27. The system of claim 24, wherein actuation of a start control initiates the electrical impulses, which continue for the predetermined amount of time.

28. The system of claim 27, wherein the start control is on a remote control unit.

29. The system of claim 21, wherein a sensor initiates the electrical impulses, which continue for the predetermined amount of time.

30. The system of claim 29, wherein a sensitivity of the sensor is programmable.
